(19) United States
(12) Patent Application Publication
(10) Pub. No.: US 2004/0006265 A1
(43) Pub. Date: Jan. 8, 2004

(54) Wireless transmission-ST-segment preserved of the standard 12 leads EKG apparatus for the remote administration of thrrombolytic therapy under severe cellular channel impairment

(76) Inventor: Karim Alhussiny, Houston, TX (US)

Correspondence Address: Lan Q. Ngo, McFatridge, Baker & Dee, P.C., Suite 730, 3900 Essex Lane, Houston, TX, 77027, US

(21) Appl. No.: 10/277,247

(22) Filed: Apr. 30, 2002

(51) Int. Cl. A61B 5/04

(52) U.S. Cl. 600/386; 600/393; 600/509

(57) ABSTRACT

In accordance with the present invention a combination of electrode patch is provided for the acquisition, detection, and compaction of the 12 leads EKG data and XYZ over a severely band limited channels such as the US cellular channels. The integrity (reduced error rates of data and maintenance of connectivity for physician and heath care professional continuously monitoring remote patient experiencing heart attack (MI)) of this data is vital to immediate decisions regarding therapy indication or contraindication as to the administration of thrombolytic therapy. Furthermore, novel High frequency relying on spatio-frequency decomposition of the averaged signal and thereby detection algorithms are also presented for early detection of ischemia and provides measures of predictive accuracies. Thrombolytic therapy is the clinical therapy used to manage acute heart attacks. Precisely, we provide an end-to-end unit comprising a patch to acquire the electro physiological signals from the individual, an acquisition device providing an event detection and compaction routines over the cellular and other band limited wireless channels and a receiver to display the data.

### BACKGROUND OF THE INVENTION

1) Organization

The disclosure is organized in the following fashion: we first discus the acquisition tools and mechanisms, we then processing salient features extractions, and finally we discuss the transmission modalities under sever channel conditions.

2) Field of the Invention

The present invention relates generally to a novel approach to mediatting potentially cardiac terminal events. Key aspect of the invention is the speed of handling a cardiac emergency anywhere any time for most of all population with utmost ease. In fact the novelty and appeal of the invention comes mainly from the speed and the ease at which the 12 standard leads is made available by the involved subject for diagnosis by remote health care professional. The invention lays the ground for new modalities for Cardiac event preemption and guarding, and more particularly to remote supervision for possibly Thrombolytic Therapy administration. The development allows for continuous and simultaneous transmission of the 12 leads and central to that is maintenance of uninterrupted sessions during crucial interval without compromising the integrity of the standard leads. Succinctly speaking the invention will save lives by providing the drivers to acquire, detect (in the case of silent events) and transmit information necessary for physician to make the immediate decision. Such drivers enable the: immediate acquisition, detection and transmission over the severely band limitted cellular channels. The invention is particularly suited for almost every one and conducive to home self-adminstration of the 12 leads ECG as apart of continouns check and balance to help preempt morbid events. Also the potential amount of savings accrued from such an implementation is unprecedented.

3) Relevant Background

While the damage to the heart muscle becomes irreversibly damaged after the first few minutes of AMI, early reperfusions of the arteries by utilizing thrombolytic therapy holds the only hope for preserving the heart muscle and reversing the damage that is indeed reversible if the therapy is administered in a timely manner. Such therapy can only be recommended for these patients if the physician has a real time 12 lead ECG data as the event is underway. While many may benefit from these medications, for others the therapy may be contraindicated for clinical reasons. It is the medication that is administered immediately upon appearance of clinical symptoms to achieve reperfusion of the clogged up arteries so that the fragile myocardium may rethrive.

The acquisition of the standard 12 leads is accomplished by a first stage single amplifier circuitry whereby all 10 standard leads are clocked through a front-end buffer and then the 12 leads are constructed. The second stage is the detection phase, which begins by decomposing the constituent leads for principal features from resolutions and scales by an array of QMF. The pertinent ECG excursions namely the P, QRS, T etc., durations in seconds and amplitudes in volts are quantified. In addition and the standard XYZ waveform is also constructed for late potential and phase activation studies. The principal components are then scanned to build a vicariate probability density function reflecting substantially more interaction (stochastically). The moments of the mass function are also obtained. A stochastic threshold (rather than a deterministic) is determined for truncating the coefficients. Self-similarity across scales with the threshold modulus being the determining factor for efficient significance. This obviates the need for multiple coefficient scanning and provides improved compression ratios beyond the well-known algorithms. Finally, channel state information (CSI) that is traditionally available from the transmitter/receiver front end is now incorporated into the quantizer to accommodate the continually ever-varying transmission capacity (rate elasticity). Current modalities for ECG transmission is limited to few leads, if at all, and insufficient for providing the ER physician with the necessary information for real time intervention. The forgoing arrangement allows for remote patient experiencing Acute Myocardial infarction and utilizing a narrow band link such as a cellular channel to be assisted by an ER physician to advice to administer or not to administer the therapy. Thrombolytic therapy, if warranted, is essential for reperfusion of occluded arteries while an ongoing heart event is unfolding. Central to this invention is making possible the provision for the Conciseness of the 12 leads and session maintenance connectivity. Early intervention and preemption viz., making available the 12 leads is key and central to the preservation of the heart muscle. Such items are crucial mechanisms enabling the proper administration of Thrombolytic Therapy. Finally, the monitor comprises routines that are assembled on demand. Physician specific permutation for statistical search over gender, age, race or the combination of any plurality of any combinations

Consequences of cardiac related events become even more pronounced when viewed from the context of their potential manifestation viz., cerebrovascular trauma. Such events may frequently be brought about by either symptomatic or silent cardiac episodes.

The potential consequence of a sever cardiac event and its impact in terms of cerebrovascular disease, more than that of any other vascular disease, lies as much with its enormous morbidity as with its substantial mortality. While cardiac emergencies continue to command the highest premium amongst all emergencies, early detection through non conventional means are becoming increasingly imperative in salvaging and in helping preserve the vital myocardium (pumping heart muscle).

Only by immediate intervention upon the onset of the potentially fatal episode, one can minimize the damage and therefore preserve both heart's functional and structural integrity.

We emphasize that the speedy administration and proper anatomical positioning of the electrodes is paramount for patients with AMI. While there have been several attempts towards designing a self contained patch for the standard 12 leads, there remains many obstacles for implementing these for real clinical settings or more importantly for patient from home. Electrodes must be located in the exact position for the 12 leads to be declared as the standard 12 leads qualifying thrombolytic therapy.

It is therefore imperative to have individually customized patch that is properly designed for the individual patient. One innovative way to accomplish that begins with the patient's first visit to medical health professional that understands and knows how to administer the 10 electrodes in the proper position. The nurse affixes the electrodes to the thorax with particular emphasis paid to the precordial electrodes. The electrodes are then marked for contrast if they are not already marked and a digital camera then takes an anterio-lateral picture of the thorax. The digital picture is now processed for the detection of the coordinates of these chest electrodes. The nurse also takes notes to the various curvatures that may be compromised by 2-D nature of the image of each patient to enhance the accuracy of the synthesized patch.

The nurse also provides, for the patient convenience, a benchmark as an easy point-of-reference where upon the rest of the electrodes must be affixed according to such point is typically chosen as the notch of the manubrial and the angle of Lewis.

A computer program then generates a properly scaled template where patient can attach to his her body at home or else where for affixing new electrode set.

The limb leads are easily and computably generated with the right arm electrode emanating and pointing 45 degrees, clockwise from the sternum. The Left arm electrode, is now pointing 45 degrees from the sternum in the opposite direction. The right leg electrode can be any where on the body and we provide multiple locations so that the induced AC and conducted may be minimized by an optimum right leg electrode or a combination. The AC component due to the 60 Hz or 50 Hz fields induce capacitive currents into the body and these capacitive or displacement currents flow in the body if the body is grounded. These currents are now called conduction currents and their drainage-out-of the amplifiers can be maximized so that very little of it appears and amplified by the amplifiers. Our experience with AC currents depicts that these currents appear in ECG recordings due to variety of reasons. Reasons like, mismatched electrode impedance, amplifiers, magnetic fields coupled with cables. While new designs of amplifiers and electrodes can reduce a good portion of the interference, further improvements remain possible such as what we are proposing herein. Clearly 60 or 50 Hz notch filtering can be applied post acquisition but significant components of the ECG may be compromised especially when we perform High resolution ECG. For typical ECG recordings and Hi-res ECG, the AC component can be minimized with proper design. Choosing the proper RL (right leg electrode) position from multiple of sights on the body can significantly reduce the drainage of the AC component. These sites vary from one individual to another; indeed they vary with the same patient and are sensitive to the position and place. Such sensitivity can be logically attributed to the fact that the fields enter the body form variety of ways and as their induced currents flow in the body in various paths. The synthesized patch can now be printed in multiple of inexpensive template copies that is customized to the individual patient.

The patient can easily attach electrodes from convenience at home in the proper medical position.

So long the patient refers and attaches the reference point on the body, the “to scale” patch can be easily, instantaneously, and more importantly administered in the proper anatomical position.

A remote physician or health care professional can now with certainty make the just and proper decision as to the indication or contraindication of the usage of the Thrombolytic Therapy.

We emphasize the causes of this invention once again, while there are several attempts that have been made towards an easily designed patch for the 12 leads, a central fact that none of these solution can provide for anatomically correct position commensurate with treatment of cardiac emergencies

The rapid acquisition and interpretation of the 12 leads electrocardiogram (ECG) remains the corner stone of the decision for or against thrombolytic therapy.

Thrombolytic therapy holds the promise of significantly decreasing the morbidity and the mortality in-patient with acute myocardial infarction (NU). The favorable effects of thrombolytic therapy can be maximized if the therapy is given promptly to an appropriate candidate. Further more, the benefit of thrombolytic therapy will be more likely sustained if careful monitoring is continued. Remote areas and those who do not have the same quality of service in the cities can mostly and significantly benefit from this invention. While the guidelines of the American Heart Associations call of immediate and continuous acquisition of 12 leads and the continuity of acquisition is crucial, current technologies do not provide for such a compelling demands. Issues related to noise and transmission and detection are central for a remote cardiac professional to make proper decisions.

Issues related to transmission capacity and whether over the standard telephone lines or over cellular. The transmission capacity of these media is limited and cannot provide for the transmission of the 12 leads in a digital secure fashion. Only high compression ratios and hence low data rate can allow for guaranteed transmission and maintenance of connectivity. It is intuitive that when the data rate of a given source is reduced, the ability of the modem to negotiate the integrity of each bit so much more enhanced. The transmitter is much more comfortable to get this bit across the channel as opposed to getting the same bit at a higher speed of signaling. This is a key requirement to maintain connectivity. It is logically intuitive that a receiver can make better decision on the polarity of a received signal (Mark or Space) if only the receiver has more time to observe this bit of information. So data sources with lower signaling rates enjoy higher chances of making it across hostile channels than higher rates. Invariably and during signaling through volatile and time varying channels, the communicating modems decide to stop negotiating the session especially for higher signaling rates. This is particularly the case when the channel is plagued by noise and received signal strength is significantly impaired due to the multiplicative fading process inherent to all cellular media. We are all too familiar with the voice quality and rate of dropping the call over the cellular link. This happens because voice command greater rates over the transmission capacity limited cellular channel. And high rates must always be negotiated to maintain the connectivity. It is usual that the receiver begin to drop too many frames due to noise and reduced signal strength in the shadowy areas. This is typical of voice quality channels that preponderates higher signaling rates commanded by the voice encoders. Typical signaling rates in the United States are 14.4 kbps and 9.6 kbps. While 12 leads EKG data can be compressed down to 1.2 kbps, maintenance of connectivity is easily attained at these rates and connectivity from modem to modem especially in environment such as these fading hostile environment can be maintained.

Life saving medical innovations and objectives must not only be designed to prolong death but should further endeavor to make feasible the means to provide quality of life. Early detection and hence immediate intervention bring reality to attaining such goals.

Devices in the existing markets badly need improvement and more importantly these devices do not provide for this much needed timely physician engagement.

Ultimately, immediate intervention is only made possible if the mobile or remote patient is equipped with portable and wireless transmission capabilities. Mobile patients rely on the existing current wireless links and their capacities in the transmission of the rather huge amount of data especially for 12 leads. A current wireless channel capacity for cellular applications is inadequate and hence a judicially designed source encoder that preserves the diagnostic integrity of the acquired 12 leads ECG is obviously paramount.

Heart rate variability is an independent startifier of morbidity with mortality. In addition, high frequency component of the QRS has been shown to have a useful predictive accuracy in assessing risk associated with Sudden Cardiac Death and due to cardiomyopathy. It is shown that these indices can provide a higher predictive accuracy than each alone. Patio-frequency analysis combined with probability distribution of the incoherent ECG rhythm constitutes the basis for both the Null and the true hypothesis. The non-synchronous nature of the Sino Atrial pace maker and upon spatial averaging exhibits unique distribution and is the basis along with the components contributed by the broadband EKG for the hypothesis.

It is the intention, amongst others, of this patent to demonstrate systemically the method by which an acquired 12 leads EKG is processed to allow for the simultaneous and real time transmission of the totality of the 12 leads without loss of signal fidelity or affecting the crucial diagnostic value.

End-to-End:

The intended end-to-end operation begins with a patient. FIG. 1 illustrates a configuration for the patch used to acquire the standard 12 leads. The key innovative features that distinguish this patch from others are the customizable positioning for the anatomical correctness by the health care professional. In addition, the patch allows frequent change by the patient of the disposable electrodes. The disclosure addresses enabling the layman to administer the 12 leads any where especially from home in the proper position. A true 12 leads require administering 10 electrodes on specific position on the chest for the 12 leads to be meaningful. While the ECG has become an exact science, its value is compromised by the improper positioning of the standard 10 electrodes. This is considerably more crucial for a patient administering to her of him self especially in an emergency situation. There have been several initiatives in what appears to be attempts to towards making an easily administrable patch but all have failed to provide the imperative proper anatomical position. Some of which touts three sizes for all and others attempted to derive 12 leads from 5 electrodes and more recently a unload that once again relies on only the chest lead but remains inconclusive about the standard position. We propose a novel a solution here where the patient relies on this customizable electrode array and reusability of the template for indefinite times. Another key feature is that the patch is equipped with multiple reference electrodes to reduce the impact of induced currents that enter the body and become conduction currents from AC and other electromagnetic radiation coupled from other sources. This is extremely crucial when the application calls for high-resolution electrocardiography. Such studies seek voltage levels that are beyond the surface electrocardiogram and in general are in the micro potential levels. The AC interference falls well within these bands of interest and can significantly compromise the classification of patient unless prevented from entering the body in the first place. While post filtering of the AC interference is a common thing after acquisition, great amount of information can be wasted with this post filtering operation for the high-resolution electrocardiography. This new patch helps reduce significantly the residual AC source without further filtering. FIG. 1 depicts the oval shape of the patch and its adjustable guides for the chest leads. It is shown how easily a nurse can prepare patient for true 12 ECG with anatomical correctness and further and more importantly, how a patient can easily remove and attach new electrodes and be in a monitor mode within less than one minute. The importance of the flexibility cannot be overemphasized.

In addition, another key objective of the disclosure is to provide, with ease, modalities enabling immediate intervention in real-time setting between patients & physicians or health care professional upon the onset of a cardiac related problem. Central to the invention is symptomatic and a symptomatic episode alarm/detection and the signaling ability over the transmission-constrained capacity of the mobile conventional wireless and landline networks. Immediate intervention & scrutiny of the culprit event by the physician is the major difference with the prevailing offline modalities of technologies related to EKG devices in the current market place. While there exists a single or there maybe as many as 6 leads available from remote but not necessarily over typical U.S. cellular links, to the author's knowledge, there has been no available implemented technology that allows for simultaneously (digital format is inherently encrypted) and real time transmission over the narrowband cellular channel. The Simultaneity and the conciseness (12 leads) is key for saving lives viz., decision of administering thrombolytic therapy for reperfusion of acutely occluded arteries.

An emergency ceases to be one if preparation is done in advance. Again, Our goal is to reduce the implication of a cardiac emergency by early detection and immediate physician intervention.

The current state of the technology market does not provide for such a service especially in the times we are witnessing and as we move forward into the twenty first century, it is only natural for us to provide such a service where the consequence is of a high premium if left unchecked.

The current state of the art in the market place to include all modes of cardiac monitoring (remote and Holter)/Arrhythmia detection/event alarming/transmission/storage/database management and cardiac search engine is extremely archaic and dangerously lacking. This is doubly troubling when viewed in light of the potential risk in terms of morbidity. The inadequacy is generally two folds and stems from lack of standardization and out dated handling and poor timely delivery of pertinent and critical data and is hence exemplified by the need for universal and unified solution. The diary manger further provide for physician arbitrarily chosen queries for any combination or permutations. Example is one where a physician or a medical student is interested in the incidence of say ST segment elevation or depression, with Bruce protocol stress test, for a certain segment of the population that may be further segmented according to gender, sex, race, medication, base line and other historical and familial pertinent data.

The proprietary medical device system will serve both physicians and patients by providing early detection and prevention of heart attacks and lethal arrhythmias. This compactly portable device worn by patient will provide physicians a real time and continuous capabilities for a simultaneous 12 lead ECG wireless transmission.

According to the American Heart Association (AHA):

1.1 Million Americans will experience a heart attack in year 2000.

650,000 will be a first-time attack and 450,000 will be a recurrent attack.

Death rate from a heart attack exceeds 30% in any given year

Estimated half of 225,000 will die within an hour of heart attacks or SCD (Sudden Cardiac Death)

12.2 Million Americans have been diagnosed with heart disease

7.2 Million are survivors of heart attacks

6.3 Million are suffering from Angina or Chest Pains.

50% of heart attacks are silent.

The American Heart Association (AHA) further confirms that immediate intervention upon the onset of ventricular fibrillation phase, a terminal arrhythmia characterized by a total electrical chaos, is most beneficial to restoring heart functional and structural integrity. The odds of survival decrease at a devastating 7% per minute once entering that phase, making the chances of survival unlikely within 10 minutes from the onset. Precursors for such terminal arrhythmia is well known and easily detected with a portable EKG unit. The window that precedes such morbid and potentially fatal episode extends well within 8 hours interval [Mirvis]. Diaries from aborted Sudden Cardiac Death (SCD) from Holter recordings depict that such markers are consistent and they invariably increase in frequency, intensity, and complexity (etiology and morphology) towards the final hour of that window. Furthermore, postmortem autopsies demonstrate that death may not be warranted in some of these cases and it occurred mainly due to electrical disturbance even though major arteries are patent or have minor occlusions. The book Heart Disease by Branwald, clearly asserts the existence of a window of opportunity wherein upon complete occlusion of a coronary artery and when promptly intervened with during this window, an otherwise significant and irreversible damage to the heart muscle can be warded off. Such a prompt intervention can only be attained and made possible by the joint invocation of all of the following processes

Acquisition (simplicity in the acquisition devices), High Resolution ECG

Processing (data compression and concentration)

Filtering (patented and first of kind stabilizers of acquired electrophysiological wave forms with utmost integrity and mitigates interference and artifacts that frequently hamper diagnostics)

Detection mechanism of injury currents resulting forms acute MI.

Detection modalities having to do with quantifying early stage post MI and due to loss of the typically upright intrisicoid across the precordial standard leads.

Inherently secure sessions that assure total privacy.

Storage (Unprecedented memory savings and low power consumption)

Transmission (Internet/cellular/cordless@home/Bluetooth and paging)

In addition to that, ease of portability is another fundamental challenge we had to meet. Further the technology allows for the first time for a physician to intervene remotely for query (from storage aboard the device) and delivery of therapy.

The proposed invention also addresses a unified and universal “All in One” device that encompasses all current functionalities and more importantly a portable and concise standard 12 leads pocket size for wireless transmission and management of the critical data in one “enveloping” unit providing functionalities equivalent to:

EKG Resting Recorders

Stress Test ECG (Clinical, Thallium etc.)

Loop/Event Recorder

Holter Monitors

Outpatient EKG units & ER ECG.

Trans Telephonic (Homecare) pre/post symptoms

Telemetry (ICU, CCU, etc.)

EKG for home Cardiac Monitoring Centers

Catheter Labs EKG

EP Labs (Noninvasive) ECG

Surgery rooms EKG

Interpretation and analysis

In addition the Heart Guardian utilizes TCP/UDP IP for the greater Internet and allows monitoring to exploit the power of the Internet.

The Internet merely allows for immediate and relatively ubiquitous and convenient access and management to this critical information. The all-in-one invention lends itself to both markets, namely, the “Classical or conventional” and the more recently “advanced” wireless and Internet based. It costs the US government $278 Billion every year to maintain patients with chronic cardiovascular disease. Many of these diseases are preventable and many of fatal episodes are preempt able and abort able. When early detection of at least that segment of the population that is considered clinically at high risk. Ultimately many will benefit from the invention and amongst those are the

Healthy Conscious (online Heart portfolio Analysis Software)

High Risk and congenitally prone (dedicated cell/PSTN & Online monitoring and physician intervention)

Ultimately provides invaluable research tools for medical students and physicians' viz. Cardiac “portal” and from intelligent databases accumulated from clinical data and diaries obtained from patients' daily routines.

The main features of the invention are two folds. First is a detection algorithm of episodic arrhythmia and silent ischemia and the second is a source encoder for the continuous and real time transmission of the 12 leads over the conventional narrow band, cellular and home telephone network.

The current disclosure presents algorithms that rely on resolving salient features of a given signal bearing information that is characterized by a finite power spectral density. The salient features here imply various signal attributes that are useful from a signal processing aspect. These aspects may be signal filtering, edge detection, pattern recognition, and presentation for entropy enhancement. The signal is decomposed into intrinsic features that are small and large and provides greater latitude in terms of signal processing viz., coefficients that easily characterize local regularity of a function or a waveform. The local regularity is in terms of localizing the transient or the random event in the spatial or frequency domains. This is of a particular interest for topics in image processing especially so for discriminating image textures for the purpose of efficient source description. Global bit allocation and assignment is key to the algorithm to accommodate the multi rate constraint. This is indeed a multi resolution (localizing fine details) that is implicitly and inherently elastic. The elasticity draws upon the ability to recognize and hence exploit insignificant coefficients branches across scales and resolutions in a structure that we have dubbed as the null trellis. Further more, the structure of this trellis inherently lends itself to further economies for video signaling viz., efficient power spectral occupancy. Our approach draws upon the simple observation that voice, biophysiological waveforms, images, speech and the like contain macroscopic structures embedded within and are time and space varying in nature. Recognition and suitable characterization of these structures is key to efficient source encoding. A prominent example of these structures are features found in image textures and. Gains in terms of efficient source representation may be attained by carefully exploiting these structures. This may be accomplished by utilizing a transform that localizes the regularity of these structures as in a voiced speech segment or an image texture. The isolated structures may be thought of as source memories and hence may be represented mathematically by functions such as correlation and may be further characterized by spectrum flatness coefficient. The region over which a correlation function (short term) may be defined constitutes local quasistationarity and hence short term local statistical stability. Where the statistical stationary here is at best is in the wide sense. The forgoing is not only limited to speech, bio physiological such as EKG and Ultra sound recovery and transmission. While zero tree like algorithm provide excellent representation and lends well to greater compression ratios, in practice, however, the value of the EZW reaches a plateau and further exploitation of the hierarchical structure may not be possible without additional refined statistical characterization of time-frequency coefficients, since (rate of distortion argument here and vector quantization business). To this end, obviously one would desire a characterization, of coarse, of the joint probability density function for the time-frequency coefficients. This total characterization in the strict sense is a luxury that may not in general be available except in some idealized cases. At a first glance, joint characterization across scales and resolutions of the analysis space appears to be difficult if not impossible. Approximation of the joint density function is therefore central to the attainments of possible source encoding gains. Inexact characterization (mainly due to nonstationarities) leads to difficulties in exploiting the interdependencies that manifest into residual memories, which in turn will represent remaining redundancies. A novel approach would be to represent this joint characterization by decomposing the joint probability space into constituent densities that embody the interdependencies across the dyadic scales. It is these interdependencies and the accurate representation of interactions amongst the decomposed coefficients allow for further encoding efficiencies and to improve transmission capacities.

Micro potential is also presented here. It has been established that signal averaging is one method to enhance the signal strength of the micropotential believed to be present during and after activation of the heart muscle. Detection of the reminance of micro Potential during the repolarization phase is believed to be an independent metric for stratifying patients with ischemia and MI for SCD. The pathogenesis of these potentials maybe attributed to fractionation due to sporadic and infracted areas within the myocardium. Such a “zig zag” propagation lends to high frequency content. Albeit small in magnitude, the micro potential can be magnified by synchronous integration of the candidate intrinscoid. Further noise elimination and perhaps classification can enhance the predictive value of the test. Fractionation due to necrotic myocardium adds to the pathological component whereas anisotropy (uniform or non uniform) adds to the physiological aspect of the micro potential. Non-uniform anisotropy can compound the high frequency micro potential and can result from enduring and chronic ischemic cells at areas near the subendoradium in the transverse direction of activation. Therefore, the high frequency behavior at the early phase of depolarization can be attributed to both physiological and path physiological mechanisms.

Clearly, fractionation can result from wave fronts being obstructed and hence locally trapped by the chronically adjacent and ischemic cells that have endured various degrees of ischemia. This raises the specter of micro sporadic gradients. While signal averaging (pulse integration in the Radar discourse) can enhance the persisting (every cycle) micro voltaic processes originating from mechanisms (pathological or non pathological) and giving contribution to a synchronous periodic may fallaciously may increase the level of the micro potential and hence resulting in false positives tests. Identification and characterization of the nature of these mechanisms viz, mathematical models can enhance the test predictive accuracy. Even early stages of subendocardial ischemia can be detected when it becomes manifestly present in terms of this micro potential and owing to such a gradient. In many instances, periodic and asynchronous noise arise form physiological sources such as breathing, AC power, EEG, etc. motors in the skeletal system.

The presence of synchronous and periodic noise can severely bias the threshold resulting in tests with limited values.

With the era of telemedicine and the morbid consequence of cardiac emergencies, it has become paramount that if technology has evolved so that, if deemed necessary by physician or as a part of healthy conscious life style, one can wear a wirelessly enabled ECG or one can be monitored when he/she is driving. Therefore with an innovative approach to continuous monitoring and with the premise of ubiquity, an important aspect of the present invention is retrofitting all cars with a convenient ecg embedded in the steering wheel. Two electrodes embedded in the clock position 2 and 10. A single instrumentation amplifier, another electrode that hooks up against any where on the left leg and the right preferably to the side of the thigh on both legs. This position is not necessary. These legs electrodes are retrofitted with blue tooth technology so that we have for electrodes at any time necessary for the six frontal leads.

Immediate intervention can now be possible as well with the advent of multi media over IP technology and mobile computing. The telecomm industry is continuously searching for improved algorithms that may fill the need for data transmission in an ever-depleting resource namely, the bandwidth. High-resolution images and high and video over IP all command high signaling rate over narrow band links.

Lower data rates effectively enable maintenance of connectivity and virtually control the physical channel in the network hierarchy. Such a feature is a central issue in session management for both landline and wireless applications. The advent of the era of thrombolytic therapy and the importance of the 12 ECG leads for administering the therapy has pushed researchers in both the bioengineering and the medical arenas to find efficient methods for acquiring the 12 leads ECG. This need is further hampered by the ever-difficult task of electrodes compliance. As a result, there have been great efforts focusing on solutions to mitigate these problems. The derived 12 leads from 5 electrodes was one of such attempts towards these milestones. It is unfortunate that the derived 12 leads have not been received well in the medical community. The reluctance towards adopting it has been mainly due its variations from the standard 12 leads. To date and even with the derived 12 leads potential lead reduction, the author is not aware of a digitally implemented technology for the transmission of the derived or otherwise standard 12 leads over narrowband air links such as the U.S. cellular links. While the author is fully aware of the compliance issue of the 10 electrodes attachment, a major breakthrough here is the ease at how the device can utilize the monitored self adhesive and patented 10 electrodes array [Stratbucker]. As shown in the Figure, an additional advantage is the reduction of base line artifacts that frequently interfere with the diagnostics especially when the patient is exercising on a treadmill.

As a consequence, the foregoing invention will preclude the proliferation of the controversially and potentially dangerous misdiagnosis based on a nonstandard 12 EKG. As mentioned earlier that the 12 leads remain to be the corner stone for the administration of Thrombolytic Therapy [Cliff, Mark, Wagner].

### SUMMARY OF THE INVENTION

According to the present invention, a portable 12 leads ekg device comprising the following features and functions that differ from the traditional form, is employed to monitor simultaneously and transmit remotely over conventional cellular and other conventional home telephone channels with a single amplifier acquiring all 8 channels.

Key differentiating feature of the present invention from previous arts is enabling the digitally continuous 12 leads transmission of 1.2 kbps to become a home appliance by the ease of administering 10 electrode patch as shown in FIG. 1 with the ability to transmit all 12 leads over the cellular in a simultaneous and continuous fashion at unprecedented low rates. The American Heart Association recognizes the importance of immediate acquisition and transmission of all leads. Not only the transmission nut maintenance of connectivity is key feature for a remote physician to monitor, make certain, o the soundness of the decision by having access to a continuous session uninterrupted. Such maintenance of connectivity can be attained by virtually controlling the physical layer by reducing the signaling rate to 1.2 Kbps. Another key feature is the elastic transmission and on demand data encoder. Such a rate variable encoding is key to multiplex other vital data along with the 12 leads EKG over the hostile, and time varying cellular channel. We insist that the ease at which an individual can administer to oneself the standard 12 leads coupled early and frequently exercised test designed for those individuals such as the healthy conscious, the congenitally prone, the high risk, the diabetic, the geographically disadvantaged and as a part of their daily routines will increase the preemption of terminal events and reduce the ever climbing cost for the chronically ill afflicted with cardio vascular disease. Coronary Artery disease remains to be the number one killer of women in the industrial world during the age of 50-60 years. The cost

1) A home appliance portable and autonomous cardiac supervisor comprising:
Means of acquisition from n electrodes through a single amplifier of the standard 12 leads and XYZ frank leads

2) An internet based algorithm with the said device for daily and routinely self-assessment at any time and or with assistance of physician protocol and internet based analysis software.

3) A vigilant supervisor preempting 90% of Sudden Cardiac Death by convenience of simple patch ubiquitously surveying and detecting new cardiac precursors such as frequent and multimorphic Premature ventricle Contractions PVC predisposing to more complex or sustained Ventricular Tachardia leading to SCD.

4) A convenient method for continuously monitoring people via 2 metallic dry electrodes in the position 2 and 10 o'clock simulating the right arm and the left arm embedded in the steering wheel of a driver with patient's thumbs gripping as he or she drives.

5) Dry or gel electrodes held conveniently against the left and right legs with blue tooth enabled wireless to provide with claim 3, the six frontal leads.

6) Similar to the claim in 3, with gel or dry electrodes may be placed on standard chest leads with blue tooth enabled and compression to allow transmission over the cellular.

7) A reduced noise electrode Array for stress testing and mobile modalities.

8) An electrode Array comprising disjoints concentric non adhesive electrodes

9) A cable interface mediating and reducing the pulling action on electrode due to muscle motion.

10) A configurable cutoff passes band analog front-end filter for 75 Hz, 100 Hz, 150 Hz, 250 Hz.

11) Analysis software provides for the individual heart portfolio as base lines and can intercept precursors that previously were not detectable in routine clinical test.

12) The device monitors all standard leads to include Frank leads for high frequency analysis as an individual goes about daily routines

13) An algorithm to stabilize baseline undulation while and due to motion.

14) An algorithm that locates the J point and J+10, . . . , J+80.

15) The device quantifies the total Ischemic burden and injury currents due to imbalances resulting form systolic and diastolic acute MI and that a score is assigned reducing all the 12 dimensionality if the EKG into a single number

16) The device increases the sensitivity and specificity of a test by normalizing the injury currents at any of the J+n points to the rate and slope at these points.

17) Immediate and simultaneous, in real time transmission over U.S narrow band cellular channels (14.4 Kbps) of the standard 12 ECG leads.

18) The invention also allows the transmission of 12 leads over the conventional cellular telephone lines utilizing the 911 Emergency services over severely narrowed and available bandwidths.

19) Method by which the portable monitor detects the fiduciary EKG points whereby the waveform is decomposed by transforms using highly regular Kernels. The synthesized coefficients in the various scales refer to the onset of these points in terms of onset and offset that determine the time duration and peak amplitude. The durations and amplitude excursions of the P, Q, R, S, J, ST, TP, QT are used to for diagnosis and interpretation of the rhythm.

20) The device detects early onset of arrhythmia and the reassuring post Throlmbolytic therapy arrhythmia. Four independent methods by which an entopic cite, such as Para systolic or premature ventricular contraction is detected. The first method utilizes the regularity of specialized functions. The specialty here refers to possession of high degrees of continuity and differentiability. Discriminatory Features are derived from the intrinsic correlations amongst the sequential series of in a barrage of normally conducted complexes. A Fourier based method in conjunction with R wave detection from the last claim and the phase excursion is the disciminant for detection. An FM third method utilizes a pulse width as a discriminant. An R wave synchronized decision directed matched filter. Another method exploits the Hotling transform in the detection of PVC from and amongst normally conducted R synchronized wave decision directed.

21) Ischemia and infarction indicators from scales depicting high energy levels when compared to isoelectric ST segments. Sensitive bands to ST deviation may be offset for populations with Normal variant (ST segment) and Juvenal type of elevated ST segment may be adjusted. The detection is equally applicable to transient Ischemia. The test shows more sensitivity if the R or the J point are localized and exact ST deviation are now quantified.

22) A tool utilizing the matrix inversion properties of the principle component transform to assess discriminates sensitive to ST segment elevation and depression.

23) Counter measures to reduce the effect of displacement currents going in to the body and becomes interfering conduction currents. The invention allows for adaptively selecting an optimum ground electrode to allow flow to ground of induced currents. Optimality here is in terms of minimizing the 60 Hz components by selecting the ground electrode that constitute the easiest (least resistive) for the displacement currents flow.

24) Quantitative tool relying on sound statistical inference techniques in assessing and interpreting results.

25) The polymorphic PVC diagnostic detector comprises of Eigen value decomposition of each PVC detected. Various forms indicate multiple sites and etiologies.

26) An adaptive cancellor for coherent AC interference and is much needed for High resolution XYX frank lead interpretations.

27) Novel wavelet based non-invasive algorithm for testing reperfusion after AMI treated patients with thrombolytic therapy and PTCA.

28) Novel noninvasive tool to test impact of anesthesia on cardiac and non-cardiac patients undergoing operations.

29) Novel analytical tool that helps assessing (to enhance the negative and positive predictive accuracies of High frequency ECG) incoherent noise owing to

30) A processor that enhances the positive predictive accuracy for cardiac micro potential tests.

31) Non invasive algorithm comprising a hybrid index exploiting jointly the reduced Heart Rate variability and broad band small features ECG in classifying Coronary Artery disease with single and multiple vessel.

32) Total ischemic burden is completely characterized over the narrow cellular band by allocating more of the significant bits to the ST segment. Localizing the R peak and the J point first does this. An on demand window is allocates the necessary bits from the corresponding detail at the appropriate scale.

33) A novel and combined index assessing the degree of coronary artery disease CAD utilizing both Heart rate variability and broadband ECG with enhanced sensitivity and specificity in one Spatio-spectral index.

34) A novel spectrally directed discriminants based on moving average of the combined synchronized QRS and HRV variations

35) A Sepeterally directed discrminants based on autoregressive model fit of a combined HRV and the averaged QRS.

36) A combined ADPCM with the implicit null trellis. Further economies are obtained from the se interactions. The quantizer is applied to a lower variance difference and hence the savings in coding.

37) Real time Algorithm that allows immediate ability for patients to receive analysis (index) over IP to patient own heart portfolio.

38) An episode detector that captures in addition to symptomatic events, a symptomatic and silent one via a single score capturing qrs width and deviation along with ST deviations from the bioelectric base.

39) Longer maninenace of sessions connectivity over cellular imperative for physician remote intervention and therapy decision for Acute Myocardial Infarction, exploiting features perception sensitive bands, exploiting unequal error protection and the volatile robust channel state information

40) Inherently encrypted for patient privacy.

41) Robust matrix inversion for principle and Eigen value Hoteling decomposition.

42) Channel identification and hence equalization using coarse scales inverse impulse equivalence.

43) Optimized length FIR unit and impulse responses interims of its invariance.

44) Indicators based on wavelet packets for ascertaining varying stationarities.

45) Reduced and multiplication free full search block matching for motion estimation.

46) An Optimized arithmetic encoder (multiplication free)

47) Source controlled encoder

48) Reduced CPU image compression.

49) Improved decoder memory manager.

50) Wavelet based SNR estimator for the channel dependent error correcting codes.

51) Combined vector quantizer and multiscale source encoder.

52) Rate controlled elasticity viz., SNR estimator.

53) SNR enhancement via a monolithic silicon and adaptive multiple antenna polarization diversity.

54) Detection of minute and settle levels of total ischemic burden of patient with cad and those that Relief of a symptomatic silent angina; Resolution reversible silent ischemic defects found upon radio nuclide assessment; Improved exercise treadmill times; Elimination or reduction of nitrate use; Ability to enjoy a more active lifestyle.

55) A threshold based on spatial stionarity rather than a deterministically decreasing one across scales and resolutions. Moments supervised thresholds provide higher economies by jointly observing the decision space.

56) Convenient N-dimensional joint density (multivariate probability density) characterization.

57) A tool preventing misdiagnosis due to the invariably and inadvertently misplaced electrodes.

58) FH and DS combination with intelligent wavelet based intruder detection and avoidance.

59) A stationarity predictor that combines wavelet packet with a moving corolometer to assess stationarity of the Random process and to attain further coding gain. Transient detector for local wide sense stationary (short term stationary).

60) Multiple electrodes for RL reference, Optimized current sink through grounding all displacement currents induced and due to 60 or 50 HZ.

61) The event recorder begins with prepping the patient. Depending on the size of that patient and physician recommendation, physician, nurse, or patient him/herself may administer the appropriate patch of three sizes with 10 electrodes per patch. The patch is easily centered on the patient body by referring to a bone marker or the “reference point” typically two inches below the Manubrial notch of the sternum. The patch contains the conductive/adhesive gel with Silver-Silver chloride electrodes and leads embedded within the lining of the patch. Patch may last for 2 weeks.

62) Two simple snaps, patient connects the RJ/10 to unit 3×2×0.5″ which snaps to small cellular unit via serial rs232 connection and a long life rechargeable spare cellular battery. The cellular battery has continuous operational session of 5 hours before it is replaced and recharged.

63) A session is established by the initiation of transmission upon a push of a button by the patient due an event occurrence experienced by the patient. A dial up precedes a transmission of configurable 15 seconds prior to action taken by the patient and 15 seconds post action upon the occurrence of the episode. The event will more likely to fall with that window with its symptomatic onset occurring prior to the action by patient time marker but well within the configurable window of typically 30 seconds duration.

64) The monitor server returns an acknowledgment of patient initiated transmission with a receipt in terms of intermittent paroxysmal beeps similar to a pager sounding beep or a short and silent vibrating shake acknowledgment to patient upon monitoring personnel approval of complete and successful session.

65) The server is also capable of a feature that exploits the duplex capability and availability of the cellular network. The monitor server can interrogates, if necessary, in cases like a lost session whereby the monitor may retrieve a valid patient record that has been initiated by the patient and that has been corrupted or never been completed without calling and disturbing the patient. The initiating event along with the post configurable duration is stored until the occurrence of newer event.

66) Feature whereupon the occurrence of certain events such as silent arrhythmia and other sustained arrhythmia that are beyond physician's prescribed thresholds, the operator, in addition to perhaps the already existing protocol of calling the patient, ER, or physician, may now signal the mobile patient over the Event cellular module. If the current protocol stipulates reaching the patient upon occurrence of certain events, such beeps may now alert the patient who otherwise might not be reachable in a prompt manner, can now benefit from such optional feature.

67) A patient using default mode of event recorder of the POTS from home or elsewhere. The cellular module is blue tooth enabled technology with a receiver base station that is commonly blue tooth enabled too. So long the patient is within several yards a way form the base station, the patient may initiate a transmission just like he or she would normally with telephone lines. The wireless capability isolates the patient from the relatively high voltages associated with these base modems connected to the wall jacks. Base stations that are Blue tooth enabled will automatically discover a blue tooth mobile user in the immediate local vicinity as part of a local Pico wireless network.

68) N roaming patients each will have his/her own cellular number suggest that the probability of two “customers” arriving independently to one server and overlapping within a time window equivalent to a total transmission session interval of one minute, over 24-hour interval is very small. The probability, however, gets even smaller as you increase the number of servers so we always have one less server than the number of the arriving customers. We feel that and with the assumption of independent events (We patients calling) and a total session from beginning to end of 1 min (service time) with twenty patients, that the expected number for line roll over (operators servicing the customers) should be no less than 5 lines (available at any time) with a probability of 99.9% of the time.

69) An N wireless 12 leads over cellular carrier neutral service handler, resolving even if the likelihood of two events occurring (two patients calling at exactly the same time) is high, a minimum of at least two lines (roll over assuming patients call same number) will preclude the occurrence of this impossible event. Also the 20 patient models assumes maximum number of patients calling every day, actual data, however, suggests it is seldom that all patents with event recorders will initiate a transmission every day, but also there is the possibility of one patient initiating more than one transmission in one day.

70) An event model assuming independent events, is intuitively logical since there is no reason why should the occurrence of a transient episode in one patient, correlate with the already “random by definition” event of another. However, the possibility of one patient having multiple events after having a single event in one particular day may not be considered all too unlikely. That is why data from previous traffic trends can help refine the estimate on the true number of lines with lower variance (higher confidence)

71) An cardiac event conservative servicing model assuming that if calls were to be made in one hour interval in that day, for some reason, as opposed to being spread logically over the 24 hrs, the required number of lines remains to be no more than N lines with a scalable confidence.

72) A model for the pharmaceutical trials may differ and hence warrant adjusting the above model slightly since the likelihood of episodes may no longer be independent and tend to correlate due to patients regiment taking medications on synchronized circadian basis.

73) A server is Windows based OS with a seamless switch over capability and data base manager over Windows NT comprising a plurality of inventions enabling
Immediate and discrete one push button transmission convenience
Provides the concise standards 12 leads needed for life saving immediate assessment
Scalable for Multimedia architecture
Designed and Optimized specifically for mobility and freedom of cellular sessions and by default to POTS
High signal fidelity with stable base line, no acoustics involved, digital coupling
Resilient to channel volatility and designed to benefit from available and proprietary algorithms
Ubiquity, neutral to all cellular carriers and precludes tolls by carrier operators for assisting non-English speaking patients.
Patient convenience, easily attachable array and the convenience of a single push button.
Inherently secured and benefits from existing encryption algorithm
Multi layered authentication protocols
Designed for real time and simultaneous transmission of the 12 leads
Immediate analysis update of recording to patient heart portfolio over IP (optional).
Courtesy and patient acknowledgment to transmission receipt.
Ability to remotely interrogate patient events and ability to alert upon device inadvertent malfunction such as an electrode becoming lose or detecting a silent ST segment by random patient polling without disturbing the patient.
